(19) United States
(12) Patent Application Publication
(10) Pub. No.: US 2023/0368900 A1
(43) Pub. Date: Nov. 16, 2023

(54) Systems and Methods for Digitally Priming a Neurodiverse Patient for Treatment

(71) Applicant: Dontsova Albina, Sapir (IL)

(72) Inventor: Albina Dontsova, Sapir (IL)

(21) Appl. No.: 17/741,726

(22) Filed: May 11, 2022

(51) Int. Cl. G16H 40/20; G16H 10/60; G16H 20/30; G16H 20/70

(52) U.S. Cl. CPC G16H 40/20; G16H 10/60; G16H 20/30; G16H 20/70

(57) ABSTRACT

An embodiment of the present invention discloses an to digitally output trigger-specific training/conditioning to at least one of the patient and/or practitioner/provider based on at least one of the patient and/or provider input. The trigger-specific output or provisioning may be at least one of a visual guide, audio guide, training video, compliance assessment, in-treatment guide, post-treatment follow-up, rewards, history, current emotional/mental state/score, or current trigger sensitivity state/score, facility retro-fitting protocol, etc.

### BACKGROUND

### Field

This invention relates generally to medical treatment solutions, and more specifically, to an automated digital tool for priming neurodiverse patients ahead of medical treatment, featuring visuals and cursor control mechanisms for enabling an at-a-glance survey of treatment provider facility triggers—not to mention further insights priming the neurodiverse patient of the triggers ahead of the treatment.

### Related Art

While current theories associating autism with an increased prevalence of oral disorders remain controversial, there remains no uncertainty regarding the association between children with Autism Spectrum Disorder (ASD) and under-met dental needs and outcomes. This is largely thought to be a result of hypersensitivity to behavioral stimuli and triggers commonly associated with a dental practice. Beyond hyper-sensitivity, another hallmark of ASD is impaired social interaction and communication, preventing further mitigation of the behavioral effects of the stimuli and reinforcing feelings of alienation. Despite not being a monolith, the ASD community does share common sensory and behavioral limitations that can affect the dental care experience necessitating a special protocol, different from that of neurotypical children. According to a recent Harvard Health Publishing article, the pediatric dental establishment has issued a de facto dental care guidance for dental care providers and patients with ASD or ASD-type sensitivities.

As with any de facto guideline, there is a lack of standardization and oversight leading to haphazard ASD dental care and care inefficiencies. The ASD protocol typically features everything from pre-visit patient-provider coordination; to pharmacological interventions, however, they are not centrally managed/automated or updated with changing provider operations and evolving patient needs. They are prohibitively taxing on the provider, who may have a practice catering to a large number of ASD patients. Many providers are already drowning in time and labor with office management—namely payroll and insurance reimbursable—and unfortunately do not have the luxury to divert resources for ASD protocol management. In one example, the protocol or guideline typically suggests the use of dimmer natural lighting in the event of an ASD patient with extreme light sensitivity. This may be addressed by simply lowering or dimming an overhead light in the waiting room or examination room, however, there are instances in which the light-emitting from a practitioner's tools (head-mounted light, for instance) may not be augmented due to manufacturer choice. As a result, a practitioner may decide to eliminate the light source, compromising patient care conditions to accommodate the patient's sensitivity. What's more, simple pre-visit coordination between the patient and the care team regarding stimuli will often result in over-looked stimuli since the pre-visit coordination does not factor in longitudinal or predictive insights from patient medical history. Simply instructing for potential triggers and the use of eyewear or ear wear during treatment to dampen the anticipated stimuli is not a comprehensive and sustainable course in stimuli management for ASD patients seeking dental treatment.

There are several digital tool kits currently on the market, training dental practitioners on ASD best practices—complete with visual assets and training videos. However, Hakama, and the like, are not specific to any one patient and only provide generic instruction for dealing with the most typical stimuli. Moreover, they do not engage the patient and provide specific details of the practitioner facility and practice features.

Therefore, there is a need for an automated system for ASD patients and dental care providers to deliver patient-specific stimuli mitigation during dental treatment. The system should factor in all of the requisite inputs from both the patient and provider to deliver hyper-specific and maximal mitigation, all while not compromising the provider's care standards. The system optionally should be able to track compliance with pre and post-treatment training, along with in-treatment routing through facility and stages of the procedure. This information could further inform care strategy, along with further feeding into the patient profile's stimuli/trigger folio. Furthermore, there is a need for applying deep learning models for constructing an interactive stimuli map of a provider's facility, allowing the patient to locate triggers and access a host of analytics and provisioning concerning the chosen trigger. Examples of provisioning could be accessing training modules for conditioning the patient of the patient and provider-specific triggers before the visit—not to mention collecting rewards and other engagement incentives, for instance.

The lack of trigger specificity in training the providers and conditioning the patients, ahead of scheduled treatment, undoubtedly leads to misaligned provisioning. There is a void in the art for an automated stimuli mitigation system and method for priming ASD patients seeking improved dental health outcomes. Further yet, there is a need for user interface functionality involving unique visual elements, routines, and cursor controls that enable quick at-a-glance analysis across analytic/diagnostic layers of information.

### SUMMARY

A system of one or more computers can be configured to perform particular operations or actions by having software, firmware, hardware, or a combination of them installed on the system that in operation causes or cause the system to perform the actions. One or more computer programs can be configured to perform particular operations or actions by including instructions that, when executed by the data processing apparatus, cause the apparatus to perform the actions. Embodiments disclosed include an automated system and method for generating an interactive trigger-rich map with a series of provisioning for conditioning neurodiverse patients for impending treatment. Further embodiments disclosed include an automated system and method for digitally priming a neurodiverse patient for improved dental outcomes.

It is an object of the invention to digitally output trigger-specific training/conditioning to at least one of the patient and/or practitioner/providers based on at least one of the patient and/or providers input. The trigger-specific output or provisioning may be at least of a visual guide, audio guide, training video, compliance assessment, in-treatment guide, post-treatment follow-up, rewards, history, current emotional/mental state/score, or current trigger sensitivity state/score, facility retro-fitting protocol, etc.

The ASD stimuli mitigation method/system may comprise an input means, processor coupled to a memory element, wherein said memory element comprises stored instruction, when implemented by the processor, causing the system to receive an input for a patient, wherein the input comprises information related to a patient healthcare event and/or at least one of a sensitivity trigger associated with said healthcare event; receiving an input from a treatment provider, wherein the input comprises information related to at least one of an interior, and/or at least one sensitivity trigger within a facility hosting said healthcare event; and generating a digital primer, wherein said primer digitally primes the patient of the at least one trigger tuned to at least one of the patient, provider, or healthcare event. The digital primer is preferably at least one of a visual or audible asset tuned to mitigate the behavioral effects of the trigger. Preferably, the asset is a training video downloaded, cached, or streamed to the patient's user device (mobile, tablet, etc.) conditioning the patient for the trigger.

It is another object to disclose a method/system to generate a digital primer, wherein said primer digitally primes the patient of the at least one trigger generic to at least one of the patient, provider, or healthcare event. The digital primer is preferably at least one of a visual or audible asset tuned to mitigate the behavioral effects of the trigger. Preferably, the asset is a training video downloaded, cached, or streamed to the patient's user device (mobile, tablet, etc.) conditioning the patient for the trigger, generically. It is believed that the ‘generic’ asset has comparable mitigative value as its ‘tuned’ counterpart. The ‘generic’ asset is any audible or visual asset of a generic version of the matched trigger, whereas the ‘tuned’ asset is the actual provider/facility-specific version of the matched trigger.

It is another object of the invention to disclose a method/system receiving an input for a patient, wherein the input comprises information related to a patient healthcare event and/or at least one of a sensitivity trigger associated with said healthcare event; receiving an input from a treatment provider, wherein the input comprises information related to at least one of a surrounding exterior, interior, and/or at least one sensitivity trigger comprising and/or within a facility hosting said healthcare event; and generating a digital primer, wherein said primer digitally primes the patient of the at least one trigger specific to at least one of the patient, provider, or healthcare event, wherein said primer comprises information and/or provisioning for at least one of: an interactive schematic of the facility layout locating each trigger, an interactive virtual tour of the facility highlighting each trigger, or a training module for acclimating the patient to each trigger, wherein the trigger is ‘generic’ or ‘tuned’.

Finally, it is an object of the invention to disclose a system/method for generating a trigger-rich interactive for a consumer, comprising the steps of: receiving an input from a consumer comprising at least one of an image, video, drop-down or text related to at least one of a service event and/or a sensitivity of the consumer-related to said service event; receiving an input from a service provider, wherein the input comprises at least one of an image, video, drop-down or text related to a surrounding exterior, interior, or at least one sensitivity trigger associated with a facility hosting said service event; and generating at least one of an: interactive schematic or tour of the facility with an icon symbolizing the at least one located trigger, a training module for acclimating the consumer to each trigger, a service provisioning status, a consumer compliance, and/or a service event/facility-route guidance (optionally, Augmented Reality-enabled) for the consumer based on the received input of the consumer and service provider.

Other aspects and advantages of the invention will be apparent from the following description and the appended claims.

### BRIEF DESCRIPTION OF THE DRAWINGS

The drawings illustrate the design and utility of embodiments of the present invention, in which similar elements are referred to by common reference numerals. To better appreciate the advantages and objects of the embodiments of the present invention, reference should be made to the accompanying drawings that illustrate these embodiments.

However, the drawings depict only some embodiments of the invention, and should not be taken as limiting its scope. With this caveat, embodiments of the invention will be described and explained with additional specificity and detail through the use of the accompanying drawings in which:

FIG. 1 illustrates in a block diagram, an exemplary networked environment of the stimuli mitigation system, according to another embodiment.

FIG. 2 illustrates in a block diagram, the stimuli mitigation system, according to yet another embodiment.

FIG. 3 illustrates an interaction flow diagram, according to another embodiment.

FIG. 4 illustrates a screenshot of an exemplary user interface, according to an embodiment.

FIG. 5 illustrates a screenshot of an exemplary user interface, according to an embodiment.

FIG. 6 illustrates an interaction flow diagram, according to another embodiment.

FIG. 7 illustrates a screenshot of an exemplary user interface, according to an embodiment.

FIG. 8 illustrates a screenshot of an exemplary user interface, according to an embodiment.

FIG. 9 illustrates an interaction flow diagram, according to another embodiment.

FIG. 10 illustrates a provisioning chart, by an aspect of the invention.

FIG. 11a depicts an interactive schematic diagram, according to another embodiment.

FIG. 11b depicts an interactive video tour, according to another embodiment. a

FIG. 12 illustrates a process flow by an aspect of the invention.

FIG. 13 illustrates a process flow by an aspect of the invention.

FIG. 14a illustrates a still-shot of a marked-up tour featuring access to a training asset concerning a matched trigger, according to an embodiment.

FIG. 14b illustrates a still-shot of a treatment/facility route guidance concerning a matched trigger, according to an embodiment.

FIG. 15 illustrates in a method flow diagram, stimuli mitigation, according to an embodiment.

FIG. 16 illustrates in a method flow diagram, stimuli mitigation, according to an embodiment.

FIG. 17 illustrates in a method flow diagram, stimuli mitigation, according to an embodiment.

### DETAILED DESCRIPTION

In the following description, for purposes of explanation, numerous specific details are outlined in order to provide a thorough understanding of the invention. It will be apparent, however, to one skilled in the art that the invention can be practiced without these specific details. Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments. These and other features and improvements of the present application will become apparent to one of ordinary skill in the art upon review of the following detailed description when taken in conjunction with the several drawings and the appended claims.

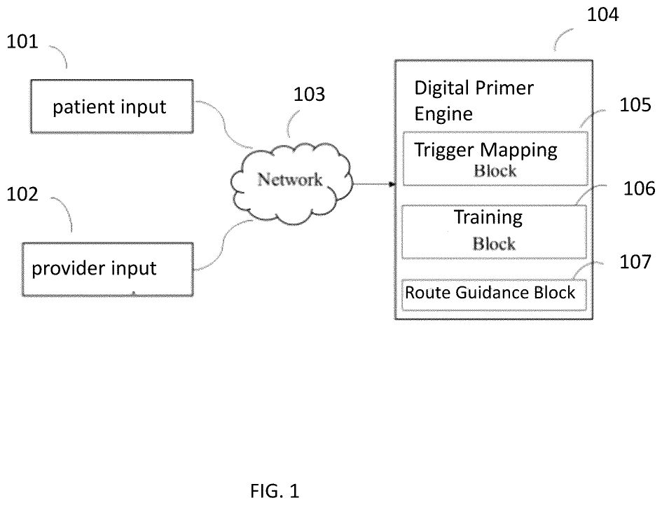

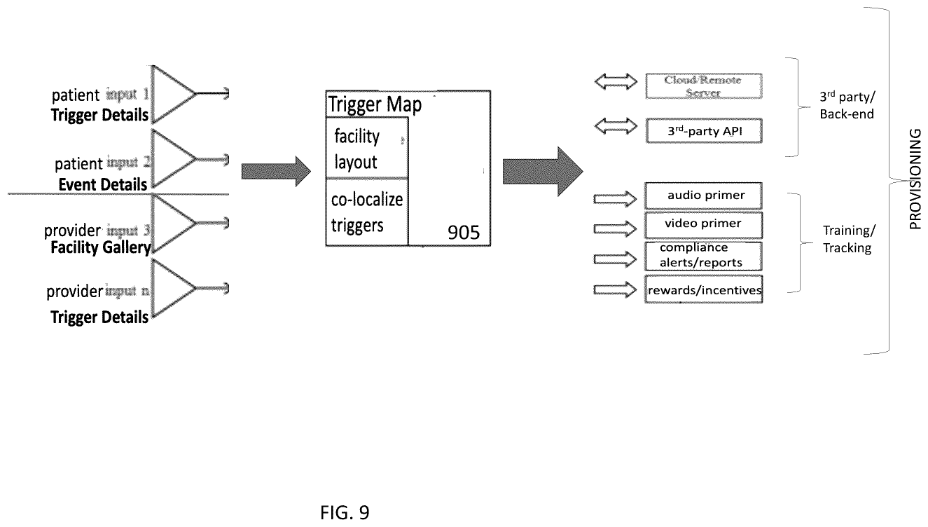

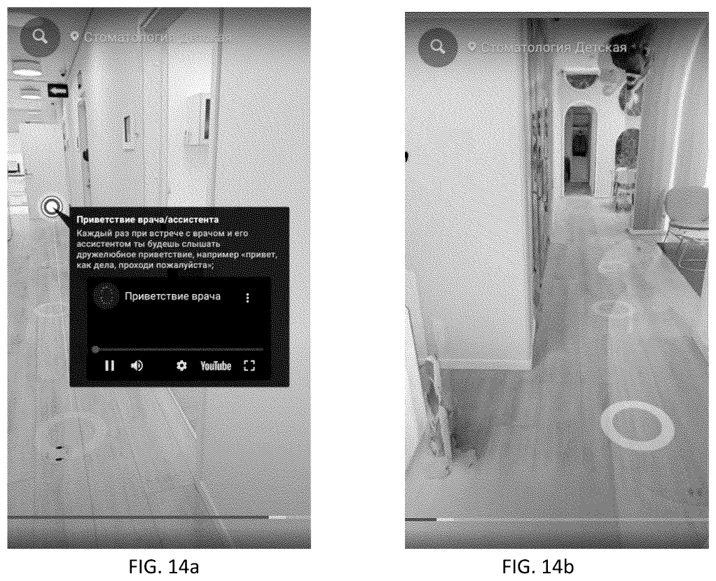

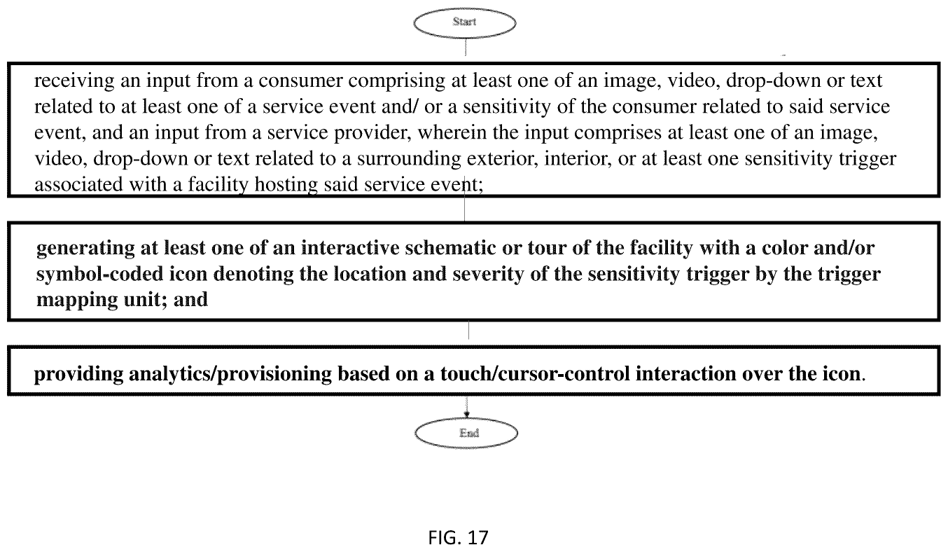

Specific embodiments of the invention will now be described in detail concerning the accompanying FIGS. 1-17. In the following detailed description of embodiments of the invention, numerous specific details are outlined in order to provide a more thorough understanding of the invention. In other instances, well-known features have not been described in detail to avoid obscuring the invention. Embodiments disclosed include an automated stimuli mitigation system and method that is specifically tuned to a specific patient and medical/dental provider facility/practice (tuned stimuli mitigation/TSM).

### Overview

The primary purpose of the disclosure is to enable an ASD patient and healthcare provider (dental/medical practitioner) to create/update a trigger profile ahead of a scheduled treatment; inform a stimuli assessment for providing patient/provider-specific desensitization or conditioning assets, and thereby lowering treatment reluctance and improving overall dental/medical care outcomes. Optionally, patient compliance may be tracked in a variety of user-device-enabled means for further defining at least one of the trigger profiles, training, or the in-treatment stimuli mitigation protocol. The value proposition of the Tuned Stimuli Mitigation (TSM) system is to alleviate the anxiety and distress associated with specific stimuli/triggers for driving improved health outcomes. For a more market-centric focus, the value proposition may apply to all links in the value chain for manufacturers, suppliers, and retailers for several different domains. The domain at focus initially is the healthcare domain—and more particularly, the dental vertical. While the dental industry has historically had high adoption barriers, the ubiquity of networked devices has led to an array of real-time, hyper-specific health assessment applications/devices for improved patient engagement—all within a pocket-reach of any tech-savvy patient. The TSM technology at the focus of the claimed invention extends the health assessment concept to include potential sensory stimuli and desensitizing patients of targeted stimuli ahead of a healthcare visit. Other consumer-based services/experiences can also capitalize on the technology—providing an assessment of potential stimuli specific to the consumer and provider facility/triggers, ahead of a consumer experience/service-provider event, etc.

### Exemplary Environment

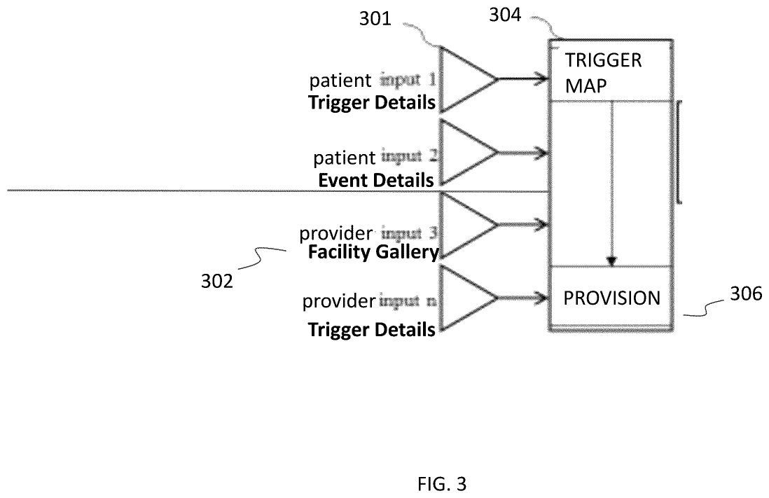

FIG. 1 illustrates an exemplary network environment and FIG. 3 illustrates an exemplary system in which various embodiments of the present invention can be practiced. The environment includes a Digital Primer Engine 104, 304 in communication with a network 103, which in turn is in communication with server/cloud, for generating a tuned conditioning asset (training 106, 306, for instance) based on the patient/practitioner input 101, 301, 102, 302, or optionally, generating an interactive trigger map 105, 304 for accessing said assets. Optionally, the environment may also provide a real-time stimuli sensitivity of the neurodiverse (ADS) patient, along with potentially tracking patient compliance to a conditioning or mitigation protocol—or even providing real-time, hyper-location based route guidance 107, 306 through a provider facility/service, featuring the precise location of trigger “hazards”, along with layered access to training/informational/“pep talk”/rewards/incentives 107, 306.

Network 103 may be any suitable wired network, wireless network, a combination of these, or any other conventional network, without limiting the scope of the present invention. A few examples may include a LAN or wireless LAN connection, an Internet connection, a point-to-point connection, or other network connections and combinations thereof. Network 103 may be any other type of network that is capable of transmitting or receiving data to/from host computers, personal devices, telephones, video/image capturing devices, video/image servers, or any other electronic devices. Further, network 103 is capable of transmitting/sending data between the mentioned devices. Additionally, network 103 may be a local, regional, or global communication network, for example, an enterprise telecommunication network, the Internet, a global mobile communication network, or any combination of similar networks. Network 103 may be a combination of an enterprise network (or the Internet) and a cellular network, in which case, suitable systems and methods are employed to seamlessly communicate between the two networks. In such cases, a mobile switching gateway may be utilized to communicate with a computer network gateway to pass data between the two networks. Network 103 may include any software, hardware, or computer applications that can provide a medium to exchange signals or data in any of the formats known in the art, related art, or developed later. The server is data storage that is configured to store data, instructions, and pre-recorded or archived videos/images. The server may be composed of a plurality of local databases or remote databases. Also, the databases may be centralized and/or distributed. In an alternate scenario, the server may store data using a cloud-based scheme.

### Digital Primer Engine:

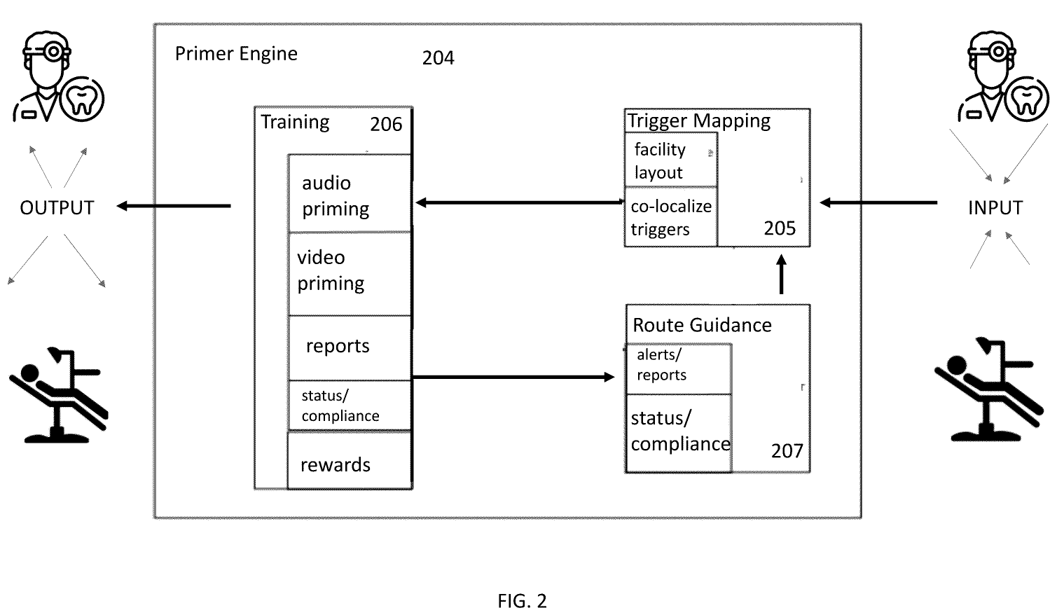

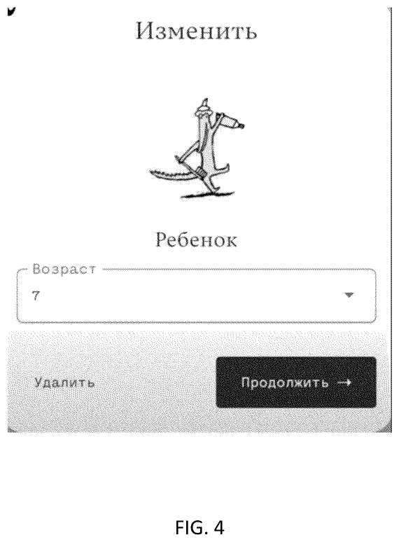

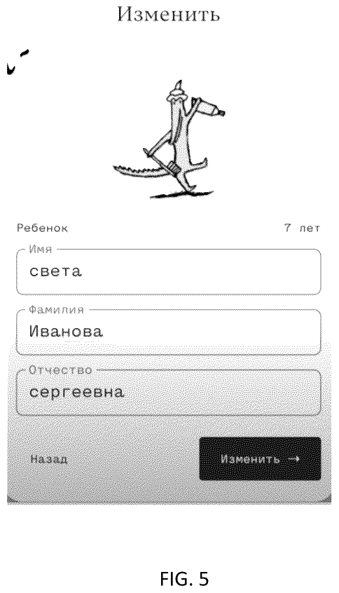

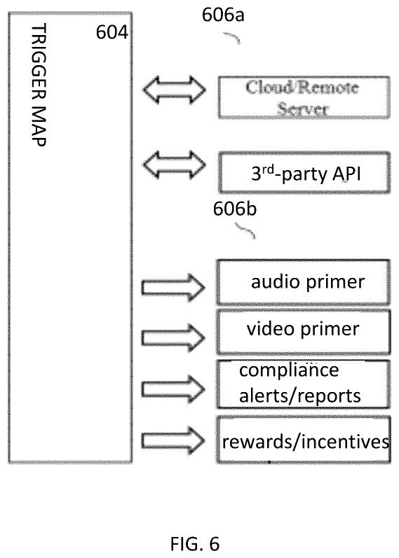

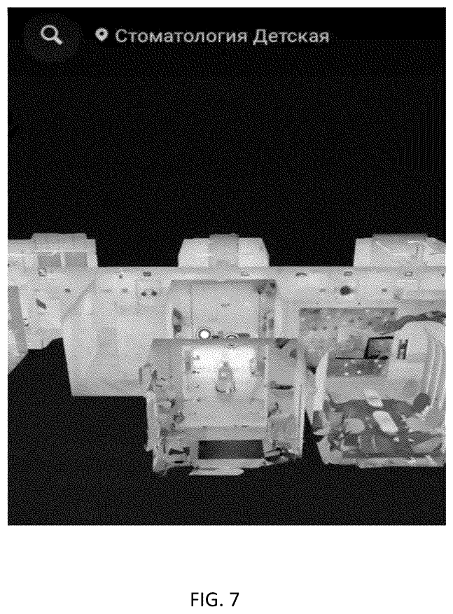

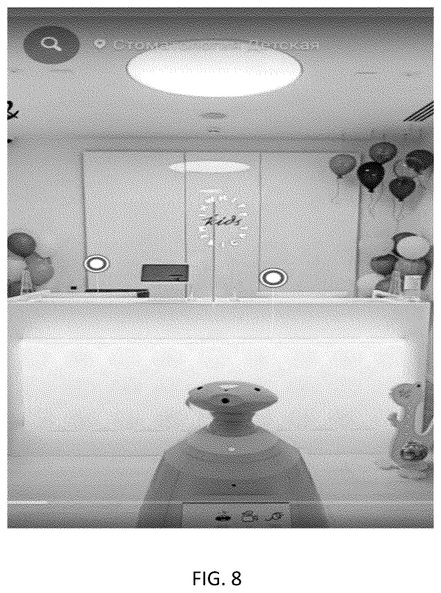

Now in reference to the figures, each illustrates a different perspective of the Stimuli Mitigation System, and more particularly, the Digital Primer Engine, its blocks, and their interrelation in achieving conditioning assets for an ASD (neurodiverse) patient ahead of treatment. Broadly, the generated assets (video, audio, map, guidance, etc.) are produced by processing at least one of the one or more received videos (or frames of videos), images, drop-down input, text input, sensor-captured input, graphically plotted—by the patient and/or provider—and executes a technique for aligning potential stimuli identified by the practitioners with the stimuli identified by patients in their respective profiles—or respective inputs. The events in the pipeline are described in the following paragraphs, with reference to the referenced figures. FIG. 2 illustrates in the block diagram, the stimuli mitigation system, according to another embodiment. FIG. 4 illustrates a screenshot of an exemplary user interface, according to an embodiment. FIG. 5 illustrates a screenshot of an exemplary user interface, according to an embodiment. FIG. 6 illustrates an interaction flow diagram, according to another embodiment. FIG. 7 illustrates a screenshot of an exemplary user interface, according to an embodiment, and FIG. 8 illustrates a screenshot of an exemplary user interface, according to an embodiment.

### Patient/Provider Input:

FIGS. 2, 6, and 9 illustrate a patient input and a medical/dental practitioner input (right-hand side) feeding into the Digital Primer Engine 204, optionally, further comprising a Trigger Mapping 205, 604, 905, Training 206, 606b, and/or Route Guidance 207. The Digital Primer Engine 204 then generates patient priming or conditioning assets for both the patient and practitioner (Output—left side). In one embodiment, a method for priming a patient comprises the steps of receiving an input for a patient, wherein the input comprises information related to a patient's healthcare event and/or at least one of a sensitivity triggers associated with said healthcare event; receiving an input from a treatment provider, wherein the input comprises information related to at least one of a surrounding exterior, interior, and/or at least one sensitivity trigger comprising and/or within a facility hosting said healthcare event; and generating a digital primer, wherein said primer digitally primes the patient of the at least one trigger specific to at least one of the patient, provider, or healthcare event, wherein said primer comprises information and/or provisioning for at least one of an interactive schematic of the facility layout locating each trigger, an interactive virtual tour of the facility highlighting each trigger, or a training module for acclimating the patient to each trigger.

In terms of provider/practitioner input, the image-capturing device (mobile device) can either have a depth camera or a regular RGB camera for capturing the precise stimuli/trigger (optionally, with surrounding context) located within a provider facility.

A provider positions the device and stands into a prompted pose/position for ideal frame capture. The prompts may be mechanical, virtual, gestured, vocal, and/or based on aligning a visual mark or box on the device screen. Video recording may either start automatically upon a correct detected position of a user, or it can be initiated by the user. The recording/capture may likewise feature a tour of the entire facility with emphasis on specific stimuli/triggers and surrounding context. Concerning patient input, a patient may record/capture potential stimuli/triggers, instead of, or in addition to, a textual/drop-down identification of triggers. Additional inputs may be achieved through any number of conventional interface techniques, such as a drop-down menu, point-plotting on a graphical display, textual input, voice-commanded, gestural, etc.

FIGS. 4 and 5 illustrate a screenshot of an exemplary user interface for engagement by the patient, caretaker, care provider, or care provider assistant. The user engages with primarily a drop-down menu for the input of patient information or provider information. Other means of input may be possible as well, such as textual input, graphical plot, etc. Patient information may be any known triggers/stimuli known by the patient, as well as the type of treatment requested from a specific provider. The patient profile may be updated upon each input, or dynamically updated based on tracked compliance to training/conditioning, and, or response to treatment. Image/video upload may be possible via the drop-down menu, wherein the user may summon a native application on the user device to retrieve and upload the image/video. Alternatively, an API-mediated tool may be exploited for layout generation. The provider may additionally upload or capture an image/video for the trigger map module of the Digital Primer Engine to render a trigger map, either a schematic of a facility layout or a 3-D virtual tour, either of which is further annotated with the location of matched triggers for further interaction. FIG. 7 illustrates a still-shot of a 3-D virtual tour of the provider facility, marking the location of a variety of stimuli/triggers, namely a brightly lit receptionist desk with a welcoming display. These particular triggers are further zoomed in on the still-shot of the frame/video of the approach to the receptionist desk in FIG. 8. Based on a cursor control, the patient may engage with the particular trigger as the patient sees fit—initiating a more granular level of detail on a need basis. In other embodiments, facility triggers are not marked on a schematic or tour, but rather provisioning simply entails providing training videos of matched triggers, specific or generic to the facility/practice.

### Patient/Provider Provisioning:

### Trigger Mapping/Training Assets:

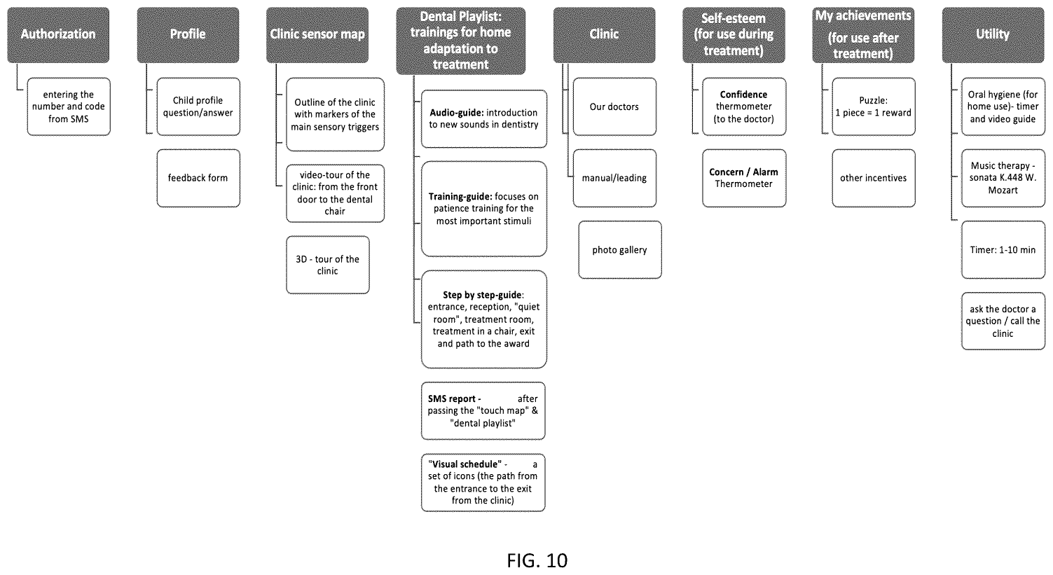

FIG. 10 illustrates the full spectrum of provisioning of the Tuned Mitigation System. Of the many categories of provisioning, the clinic sensor map refers to the trigger mapping earlier discussed—both a marked-up layout schematic and/or a virtual tour. Also, FIG. 10 highlights a “Dental Playlist”, referring to the host of provisioning beyond, or in replacement of, the trigger map/clinic sensor map, including an audio/video training guide, sensor-enabled compliance tracking, reports, or facility/treatment route guidance.

After passing all the stages of the lesson, an SMS report is sent to the clinic/doctor (the program automatically analyzes the complete passage of all levels—a sensory map, a virtual journey, training) about the child's readiness to interact with the dentist (the program collectively evaluates the data on the date of registration, the date of completion of the study of the occupation of the stage, the duration of the non-stop passage of the path and the 3D tour, the duration or number of repeated passage of the most problematic sections, the frequency of pressing the stop button by the user).

The child gradually becomes familiar with the use of various media (graphics, text, video, photography, animation, sound) with all types of stimuli that are available in the dental clinic (audio, video, olfactory, gustatory and tactile). It does this by examining a “clinic sensory map,” a three-dimensional diagram of a clinic that shows areas of sensory overload and relaxation. When studying this map, you can find out detailed information about all stimuli (types of sensory overload are marked with special symbols) by clicking on the stimulus symbols: visual stimuli are displayed on the video with the possibility of stopping, and sound stimuli are displayed. in audio format, the video shows the doctor's touches (tactile overload), etc. After the child is familiar with all the stimuli in the clinic, you can move on to the next stage—the study of the “Dental Playlist”, which includes 3 consecutive guides:

Audio-guide: a video introducing new sounds that the child will hear during dental treatment (the sound of a drill, the sound of an air jet from the dashboard of the dental chair, the sound of metal instruments in a metal instrument container, sound when the dental chair moves, the sound of a sterile craft bag being torn apart-paper, etc.).

Step-by-step guide: It has voice guidance and represents the route from the entrance to the clinic to the exit from the clinic after treatment (contact with the administrator, contact with children's waiting areas, “quiet room”, toilet interior, all the way to the treatment room, the treatment room itself, the main stages of treatment in a chair, leaving the room and the path to the reward During the demonstration of the video, a hint for parents appears on the screen from time to time with a recommendation to “give the child what he loves” (food, tactile and other reinforcement). (by clicking on the light icon) when the child sounds the alarm.

When re-passing the route and there is no alarm in the previously problematic area, the parents should turn off the “glowing light”.

The step-by-step guide is no more than 4 minutes long and is divided into segments of 20 seconds. It is recommended to use the guide in stages, gradually, after getting used to and without anxiety, adding another segment for 20 seconds.

Training-guide: focused on training patience with the inevitable stimuli during treatment, contains a different number of levels (from 1 to 10). The number of levels determines the quality of stimuli that are important for treatment (for example open mouth, motionless position during X-ray diagnostics, examination, anesthesia, tooth preparation, filling, etc.). A GIF stimulus, such as an open mouth, is displayed on the screen, and a timer starts at the bottom. By default, throughout the training, music is turned on (sonata K. 448 by W. Mozart) to improve cognitive functions in patients with mental disorders, including those with ASD, while listening to this piece of music. This phenomenon was first described as the Mozart effect in 1993 by F. Raussher et al. In the process of such training at home, the child can open his mouth and sit quietly, but when patience runs out, press the stop. Each stimulus has its own “limit/norm of patience”, for example, examination of the oral cavity is possible intermittently because during the dental examination you can close your mouth. This stimulus can be set to a minimum level, and the desired time can be set as 5-10 seconds, and so on. Each stimulus is a specific requirement for the child (do not close your mouth, do not move). After passing all the levels—a reward, positive reinforcement.

After passing the above stages, an SMS report is sent to the clinic/doctor about the readiness of a child with ASD to see a dentist (the program collectively evaluates data on the date of registration, the date of completion of the study of guides, the duration of overcoming the dental playlist of guides, the duration of the passage of the most problematic zones). Before sending the report, the parent/guardian writes the codeword/stop on a special line. If a child utters this word, the doctor must stop treatment.

The previously listed steps are partially duplicated in the “Visual Schedule” section, which contains a set of icons that illustrate the path from the entrance to the exit of the clinic.

In the course of treatment, the doctor can use the diagnostic segment of the application based on the method of visual/verbal analog scales—“trust thermometer (to the doctor)” and “alarm thermometer”. The child himself, at the request of the doctor, by pressing his finger on the screen of the gadget, selects the symbol corresponding to his emotional state.

The tendency to methodical “gathering”, as a feature of the psyche of children with ASD, is realized in the section “My achievements”, which is recommended to be used after successful treatment as positive reinforcement. With the permission of the doctor, the child can activate/open one segment of the puzzle, each of which makes up a picture from the “special interest” of the child with ASD. On the completely “assembled” puzzle, in addition to the image, there is an invigorating inscription—parting words (for example, good, very good, you have defeated caries, etc.). A library of images (available only to parents or a doctor), from where the parent can select pictures for future puzzles by the special interest of the child (trains, elevators, flowers, insects, airplanes, etc.).

The last section of the application is represented by utilities: oral hygiene assistant, music therapy with Mozart's sonata, timer, and question-answer (communication with the clinic).

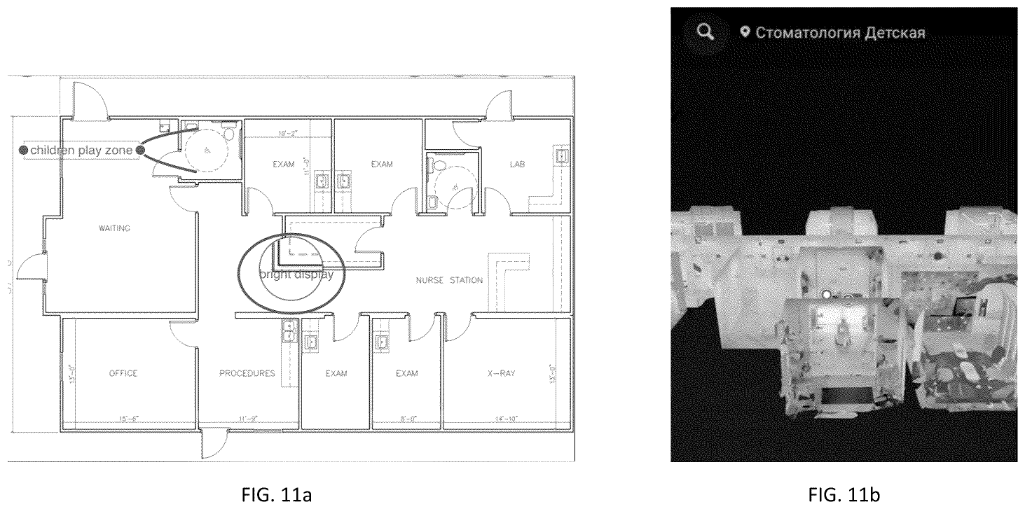

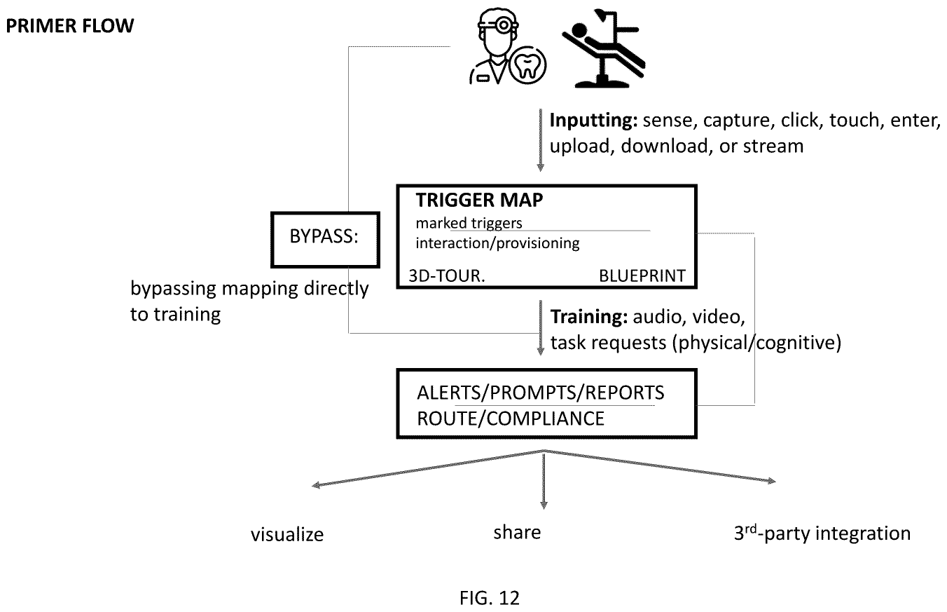

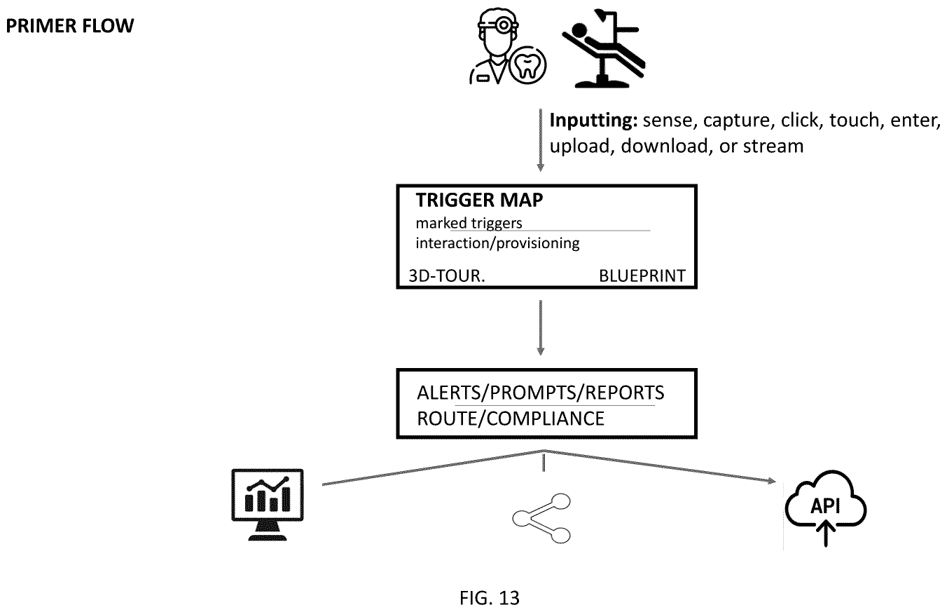

FIGS. 11a and 11b illustrate an exemplary facility layout schematic with marked triggers, and a marked-up 3-D facility tour, respectively. FIG. 11a marks the location of the “brightly lit display” near the receptionist desk in schematic form, while 11b marks the “brightly lit display” in a front-facing “POV” perspective. In some embodiments, the layout may be interactive, allowing for a patient to cursor hover over the marked trigger to reveal certain information and, or downstream access to conditioning assets (video, audio training, etc.). As illustrated in FIG. 12, which is an exemplary process or decision flow of the Digital Primer Engine or Tuned Stimuli Mitigation system, the Digital Primer Engine may bypass the interactive facility schematic, and just generate a training asset. FIG. 13 illustrates a process flow involving the generation of the marked facility schematic for downstream provisioning. The training asset may be patient and provider-specific (tuned), in which the training asset features the precise stimuli/trigger—as it appears/functions in the context of the specific provider facility for the chosen treatment. Alternatively, the training assets may not be tuned, and simply deliver training for the trigger, albeit not specific to the actual provider facility trigger.

The trigger map may be natively generated by summoning a native sketch tool and/or a third-party sketch tool for enabling a touch-input sketch of a layout of the facility, including for at least one of an orientation and general proportion of rooms, safe havens, access points, or location of known and/or potential triggers. The physical sketch is at least one of an upload of a hand-sketch and/or any file related to the layout of the facility, including at least one an orientation and general proportion of rooms, safe havens, access points, or location of known and/or potential triggers. The interactive layout schematic and/or the virtual tour may enable a user or the patient to access assets associated with marked-up triggers or further mark up an object as a known or potential trigger other than the initially marked-up map.

The icons may be further coded by color and/or symbol to further convey a risk assessment score or stimuli score. For instance, a heavier and bolded icon of a light bulb proximal to the receptionist's desk may suggest a higher severity of stimuli risk in comparison to other marked-up triggers. This further helps in prioritizing training/conditioning assets/protocol.

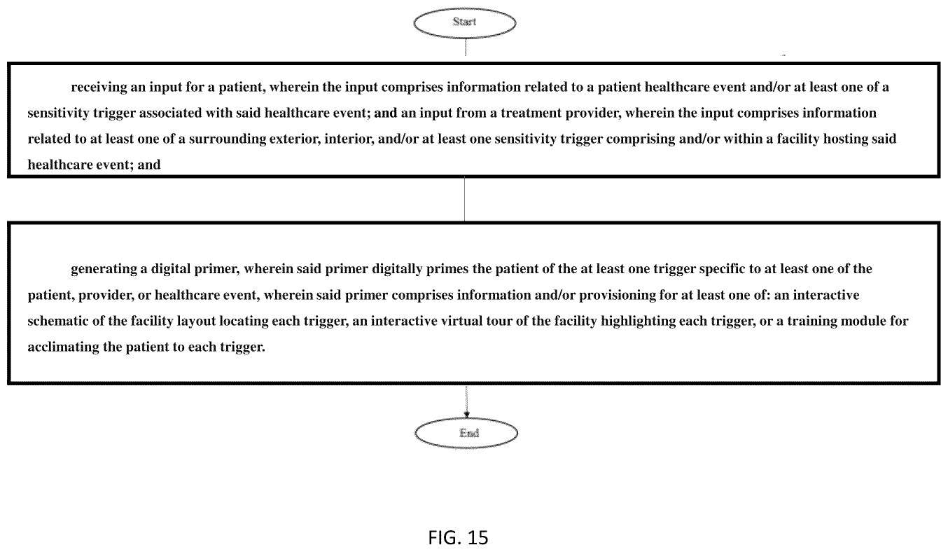

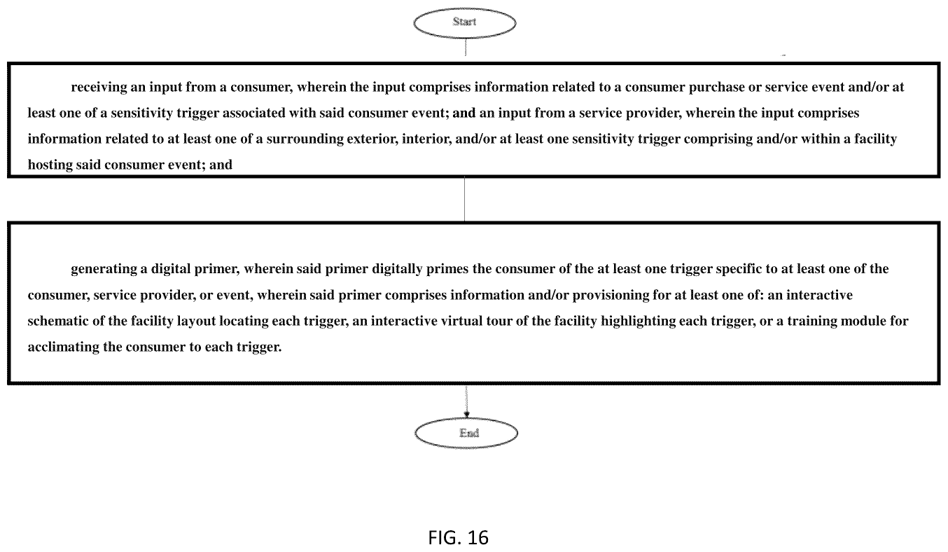

Alternatively, the conditioning assets generated may be derived from a consumer and service provider input of potential consumer-related stimuli/triggers. In one embodiment, as illustrated in FIG. 15, an exemplary method diagram, the method for generating a trigger-rich interactive for a consumer may comprise the steps of: receiving an input from a consumer comprising at least one of an image, video, drop-down or text related to at least one of a service event and/or a sensitivity of the consumer-related to said service event 1502; receiving an input from a service provider, wherein the input comprises at least one of an image, video, drop-down or text related to a surrounding exterior, interior, or at least one sensitivity trigger associated with a facility hosting said service event 1504; and generating at least one of an: interactive schematic or tour of the facility with an icon symbolizing the at least one located trigger, a training module for acclimating the consumer to each trigger, a service provisioning status, a consumer compliance, and/or a service event/facility-route guidance for the consumer based on the received input of the consumer and service provider 1506. As illustrated in FIG. 16, another method diagram by an aspect of the invention details the steps for the Digital Primer Engine generating a tuned or non-tuned conditioning asset, namely an audio/video training asset, without a trigger-mapped facility schematic with downstream provisioning. Essentially, the Engine is mapping the patient-selected potential stimuli with provider-selected potential stimuli specific to the facility/treatment, to deliver curated trigger-conditioning support in the form of a video tutorial, etc.

Although specific terms are employed, they are used in a generic and descriptive sense only and not for purposes of limitation, the scope of the invention being defined by the following claims. Those skilled in the art will recognize that the present invention admits to a number of modifications, within the spirit and scope of the inventive concepts, and that it may be applied in numerous applications, only some of which have been described herein. It is intended by the following claims to claim all such modifications and variations which fall within the true scope of the invention.

Embodiments described in the present disclosure can be implemented by any system having a processor and a non-transitory storage element coupled to the processor, with encoded instructions stored in the non-transitory storage element. The non-transitory storage element can be accessed by a general-purpose or special purpose computer, including the functional design of any special-purpose processor. A few examples of such non-transitory storage elements can include RAM, ROM, EEPROM, CD-ROM, or other optical disk storage or other magnetic. The processor and non-transitory storage element (or memory) are known in the art, thus, any additional functional or structural details are not required for the current disclosure.

We claim:

1. A method for priming a patient, said method comprising the steps of:
receiving an input for a patient, wherein the input comprises information related to a patient healthcare event and/or at least one of a sensitivity triggers associated with said healthcare event;
receiving an input from a treatment provider, wherein the input comprises information related to at least one of a surrounding exterior, interior, and/or at least one sensitivity trigger comprising and/or within a facility hosting said healthcare event; and
generating a digital primer, wherein said primer digitally primes the patient of the at least one trigger specific to at least one of the patient, provider, or healthcare event, wherein said primer comprises information and/or provisioning for at least one of: an interactive schematic of the facility layout locating each trigger, an interactive virtual tour of the facility highlighting each trigger, or a training module for acclimating the patient to each trigger.

2. The method of claim 1, wherein the patient input comprises at least one of a text, touch, gesture, drop-down, image/video capture, sensor-capture, or graphical plot of at least one of a patient profile information, a current assessment of mental and/or physical state of the patient, a current request for a healthcare event by the patient, or facility information expected to provide said healthcare event).

3. The method of claim 2, wherein the patient profile information comprises at least one of a medical history, biographical information, known and/or potential sensory triggers.

4. The method of claim 1, wherein the provider input comprises at least one of a text, touch, gesture, drop-down, image/video capture, sensor-capture, or graphical plot of at least one of an image and/or video capture of provider facility, image and/or video capture of provider facility triggers (known and/or potential), information related to the facility and triggers, or a digital and/or physical sketch of a layout of the facility.

5. The method of claim 4, wherein the digital sketch is natively generated by summoning a native sketch tool and/or a third-party sketch tool for enabling a touch-input sketch of a layout of the facility, including for at least one of an orientation and general proportion of rooms, safe havens, access points, or location of known and/or potential triggers.

6. The method of claim 4, wherein the physical sketch is at least one of an upload of a hand-sketch and/or any file related to the layout of the facility, including for at least one of an orientation and general proportion of rooms, safe havens, access points, or location of known and/or potential triggers.

7. The method of claim 1, wherein the interactive layout schematic and/or virtual tour enable a user or the patient to further mark-up an object as a known or potential trigger other than the initially highlighted triggers upon being generated.

8. The method of claim 7, wherein the marked-up and/or highlighted trigger is interacted with via touch or cursor control for information and/or provisioning related to the trigger.

9. The method of claim 1, wherein the information and/or provisioning is accessed by a search or drop-down function.

10. The method of claim 1, wherein the information and/or provisioning further comprises at least one of a: training status, treatment compliance, in-treatment program, after-treatment program, incentives, and/or a facility/event-route guidance for the patient.

11. The method of claim 10, wherein the training and/or treatment compliance is further measured based on a sensor-captured or timed response to a physical and/or cognitive task request.

12. The method of claim 1, wherein the training modules are calibrated in terms of types and extent of training is based on a scored risk assessment of the trigger.

13. A method for generating a trigger-rich interactive for a consumer, said method comprising the steps of:
receiving an input from a consumer comprising at least one of an image, video, drop-down or text related to at least one of a service event and/or a sensitivity of the consumer related to said service event;
receiving an input from a service provider, wherein the input comprises at least one of an image, video, drop-down or text related to a surrounding exterior, interior, or at least one sensitivity trigger associated with a facility hosting said service event; and
generating at least one of an: interactive schematic or tour of the facility with an icon symbolizing the at least one located trigger, a training module for acclimating the consumer to each trigger, a service provisioning status, a consumer compliance, and/or a service event/facility-route guidance for the consumer based on the received input of the consumer and service provider.

14. The method of claim 13, wherein the icons are color and/or symbol-coded to denote a severity of the trigger.

15. The method of claim 13, wherein the icons are size-coded to denote a severity of the trigger.

16. The method of claim 13, wherein the training modules are reconfigurable based on the consumer preference for training.

17. A system for generating a trigger-rich interactive for a consumer, said method comprising the steps of:
a trigger mapping unit:
a processor;
a memory element coupled to the processor;
said memory element with encoded instruction, when implemented by the processor, cause the system to:
receive an input from a consumer comprising at least one of an image, video, drop-down or text related to at least one of a service event and/or a sensitivity of the consumer related to said service event;
receive an input from a service provider, wherein the input comprises at least one of an image, video, drop-down or text related to a surrounding exterior, interior, or at least one sensitivity trigger associated with a facility hosting said service event; and
generate at least one of an interactive schematic or tour of the facility with a color and/or symbol-coded icon denoting the location and severity of the sensitivity trigger by the trigger mapping unit.

18. The system of claim 17, wherein the icon is configured to reveal varying access to information and/or provisioning based on the cursor or touch control of the consumer.

19. The system of claim 18, wherein the consumer compliance with training, service provisioning, or consumer route is alerted and/or reported to the consumer and/or service provider upon deviation from a norm.

20. The system of claim 17, wherein the trigger mapping unit co-locates the location of a trigger on the schematic and/or tour based on the provider input of facility layout schematics and consumer/provider triggers using any one of deep learning technique.
